(19) United States
(12) Patent Application Publication
(10) Pub. No.: US 2025/0222050 A1
(43) Pub. Date: Jul. 10, 2025

(54) Chlorella and Corn Leaf Extract Compositions, Their Production, and Methods of Use

(71) Applicants: Unigen, Inc., Tacoma, WA (US); Chlorella Industry Co., Ltd., Minato-ku Tokyo (JP)

(72) Inventors: Mesfin Yimam, Tacoma, WA (US); Ping Jiao, Tacoma, WA (US); Qi Jia, Tacoma, WA (US); Takayoshi Minagawa, Fukuoka (JP); Tsuyoshi Fukunaga, Yokohama Kanagawa (JP); Toshihiro Kanno, Mutsumonmachi Kurume-shi Fukuoka (JP)

(73) Assignees: Chlorella Industry Co., Ltd., Minato-ku Tokyo (JP); Unigen, Inc., Tacoma, WA (US)

(21) Appl. No.: 18/406,580

(22) Filed: Jan. 8, 2024

(51) Int. Cl. A61K 36/05; A61K 31/538; A61K 45/06; A61P 25/20

(52) U.S. Cl. CPC A61K 36/05; A61K 31/538; A61K 45/06; A61P 25/20

(57) ABSTRACT

Compositions are disclosed that include a plant extract standardized with an amount of benzoxazinoids, wherein the composition additionally comprises Chlorella. In contemplated embodiments, these compositions are provided in an effective amount to shorten sleep onset latency, improve sleep quality and efficiency, and prevent and treat sleep disorders.

### FIELD OF THE SUBJECT MATTER

The subject matter disclosed herein includes compositions of chlorella and corn leaf extract, along with their production and methods of use, including their use to reduce sleep latency and improve sleep quality.

### BACKGROUND

Poor sleep quality manifested in the form of increased sleep onset latency, decreased deep sleep duration, decreased deep sleep time and total sleep time, fragmented nighttime sleep, frequent nocturnal awakenings, increased time spent awake after sleep onset are key factors for adverse effects on psychological state and mental health. Clinically validated dietary supplements with known mode of action can be considered as a favorable alternative to conventional pharmaceutical drugs because of their safety.

Chlorella is a type of green algae that has gained popularity as a nutritional supplement because of its rich nutrient content. While it's primarily known for its potential benefits for overall health and implied for sleep support, there isn't substantial scientific evidence specifically linking chlorella to any specific sleep latency, sleep time and sleep quality improvement.

Zea mays (corn) leaf extract standardized at 6-MBOA on the other hand is known to increase the deep sleep time and total sleep time in human while it increased the incidence of falling sleep and sleep duration in animals but not known to shorten sleep latency in either of the cases.

Melatonin, which is known to the factor of shorten sleep latency, is synthesized from tryptophan. So that, the amount of synthesized melatonin is dependent on the abundance of tryptophan. 6-MBOA is known for activation of AANAT which is one of the enzymes for melatonin synthesis. Accordingly, if tryptophan is depleted, pathway of melatonin synthesis is stopped, and it will cause for lower level of sleep effect. Thus, an increase in tryptophan consumption will prolong the duration of effect of Melatonin. More than 60% of dry weight of Chlorella is occupied by protein which is rich in essential amino acid with tryptophan content at 1.1% in Chlorella. In addition, rich in micronutrients which play a role in sleep. For example, vitamin B12 for regulatory factor of circadian rhythm, folic acid for coenzymes of synthetic pathway of melatonin, carotenoid and PUFA for regulation of sleep, and so on are listed (Non-Patent Literature, 1&2). In particular, vitamin B12 is lacking in plant-based foods and chlorella is known as a plant-based ingredient which is rich in vitamin B12. Nutritional composition for characteristic feature of chlorella, chlorella intake could be complementary or synergistic with getting adequate sleep.

To the best out our knowledge, this is the first time where Chlorella alone (the contemplated materials as described herein) or combination with Zea mays (corn) leaf extract standardized at 6-MBOA has been investigated to show scientific evidence for sleep support effect.

### SUMMARY OF THE SUBJECT MATTER

Compositions are disclosed and discussed herein that include a plant extract standardized with an amount of benzoxazinoids, wherein the composition additionally comprises Chlorella.

In contemplated embodiments, these compositions are provided in an effective amount to shorten sleep onset latency, improve sleep quality and efficiency, and prevent and treat sleep disorders.

In other contemplated embodiments, plant extracts standardized with an amount of benzoxazinoids are extracted, enriched, and standardized from a plant species selected from the group comprising Zea mays, Chlorella sorokiniana, Chlorella vulgaris, Oryza species, Oryza sativa, Oryz glaberrima, Oryz australiensis, Oryz brachyantha, Secale cereale, Acanthus arboreus; Acanthus ebracteatus, Acanthus illicifolius, Acanthus mollis, Avena sativa, Avena abyssinica, Avena byzantine, Avena nuda, Avena strigosa, Hordeum vulgare, Coix lachryma-jobi, Triticum aestivum, Triticum compactum, Triticum sphaerococcum, Triticum turanicum, Sorghum bicolor, Agropyron repens, Blepharis edulis, Balsamocitrus paniculate; Peristrophe roxburghiana; Strobilanthes cusia; Lamium galeobdolon, Lobelia chinensis, Leymus chinensis, Aphelandra spp, Scoparia dulcis, Capparis sikkimensis ssp, or a combination thereof.

### BRIEF DESCRIPTION OF THE FIGURES

FIG. 1 shows contemplated tablets comprising chlorella and Zea mays (corn) leaf extract, as described in Example 14.

### DETAILED DESCRIPTION

Compositions are disclosed and discussed herein that include a plant extract standardized with an amount of benzoxazinoids, wherein the composition additionally comprises Chlorella. In contemplated embodiments, these compositions are provided in an effective amount to shorten sleep onset latency, improve sleep quality and efficiency, and prevent and treat sleep disorders.

In other contemplated embodiments, plant extracts standardized with an amount of benzoxazinoids are extracted, enriched, and standardized from a plant species selected from the group comprising Zea mays, Chlorella sorokiniana, Chlorella vulgaris, Oryza species, Oryza sativa, Oryz glaberrima, Oryz australiensis, Oryz brachyantha, Secale cereale, Acanthus arboreus; Acanthus ebracteatus, Acanthus illicifolius, Acanthus mollis, Avena sativa, Avena abyssinica, Avena byzantine, Avena nuda, Avena strigosa, Hordeum vulgare, Coix lachryma-jobi, Triticum aestivum, Triticum compactum, Triticum sphaerococcum, Triticum turanicum, Sorghum bicolor, Agropyron repens, Blepharis edulis, Balsamocitrus paniculate; Peristrophe roxburghiana; Strobilanthes cusia; Lamium galeobdolon, Lobelia chinensis, Leymus chinensis, Aphelandra spp, Scoparia dulcis, Capparis sikkimensis ssp, or a combination thereof.

Specifically, as disclosed herein, compositions containing Zea mays leaf extract and Chlorella, a green algae, have a synergistic effect in improving sleep latency and sleep quality. In contemplated embodiments and as will be disclosed herein, the combination of Chlorella and Zea mays (corn) leaf extract standardized at not less than 0.2% 6-MBOA demonstrated statistically significant shortening of sleep onset latency indicating the unexpected synergistic benefit of combining these two materials. The sleep onset latency time for the combination reduced to 33.0+/−8.9 min. compared to 44.0+2.1 min. and 35.4+16.0 min. for Zea mays (corn) leaf extract standardized at 6-MBOA and Chlorella, respectively, when administered alone. The combination showed 25% and 6.8% reductions in the time required to fall asleep when compared to Zea mays (corn) leaf extract standardized at 6-MBOA or chlorella given alone, respectively. These complementary effects of Zea mays (corn) leaf extract standardized at 6-MBOA and Chlorella was also demonstrated by up to 44.4% reductions in the standard deviation minimizing the variations in benefits between individuals when administered the combination than Zea mays (corn) leaf extract standardized at 6-MBOA or chlorella alone. The reduction in sleep onset latency was also compared against the melatonin as the positive control group and it was found that the combination of Zea mays (corn) leaf extract standardized at 6-MBOA (250 mg/kg) and chlorella (5000 mg/kg) produced statistically significant reduction in sleep onset latency when compared to melatonin.

Unexpected synergistic effect in increasing incidence of falling asleep was observed when Zea mays (corn) leaf extract standardized at not less than 0.2% 6-MBOA was combined to chlorella. When mice were administered with only Chlorella at 1000 mg/kg produced statistically non-significant 47% sleep incidence. On the other hand, the addition of 250 mg/kg Zea mays (corn) leaf extract standardized at 6-MBOA to chlorella at this dosage produced statistically significant 53% sleep incidence. At the highest dosage tested, the sleep incidence for chlorella was 60% and 53% for Zea mays (corn) leaf extract standardized at 6-MBOA, this percentage was increased to 73% for the combination because of the addition of Zea mays (corn) leaf extract standardized at 6-MBOA highlighting the unexpected synergistic effect of these materials. There were a 20% and 13% increase in the sleep incidence for Zea mays (corn) leaf extract standardized at 6-MBOA and Chlorella, respectively, when compared to their combination. Sleep incidence response for the chlorella group was not dose correlated when administered alone at 1000, 2000 and 5000 mg/kg. The highest sleep incidence (67%) was observed for the mid dose (2000 mg/kg). The addition of Zea mays (corn) leaf extract standardized at 6-MBOA at 250 mg/kg at each dosage produced a dose correlated 53%, 60% and 73% sleep incidence response.

Inverse correlation in the duration of sleep and Chlorella dosage was observed. The highest duration in sleep was observed for the low dose of Chlorella. The addition of Zea mays (corn) leaf extract standardized at 6-MBOA did not further increase sleep duration. A range of 59.6-62.6 minutes in sleep duration was observed for the combination of Chlorella and Zea mays (corn) leaf extract standardized at 6-MBOA. These findings were within the ranges of Chlorella at high dose (5000 mg/kg) or Zea mays (corn) leaf extract standardized at 6-MBOA (250 mg/kg) administered alone which were 61.6+/−16.3 and 63.5+/−10.8 minutes, respectively. The addition of Zea mays (corn) leaf extract standardized at 6-MBOA to Chlorella resulted in duration of sleeps equivalent to the melatonin treated mice for all the dosages. Zea mays (corn) leaf extract standardized at 6-MBOA by itself produced a duration of sleep similar to that of melatonin. Zea mays (corn) leaf extract standardized at 6-MBOA has been clinically proven to increase the quality of sleep. While the stages of sleep were not determined in this study, the impact of Zea mays (corn) leaf extract standardized at 6-MBOA is on the quality of sleep than quantity.

Anecdotal sleep supporting property of Chlorella has been reported. This effect could possibly be linked to the level of tryptophan. The minimum dosage of tryptophan as a nutritional supplement reported to have an impact on sleep was 250 mg/day in humans (Sutanto et al., 2022). According to the FDA guidelines this is an equivalent dosage of 4.2 mg/kg in human which will lead to 50 mg/kg mouse dose (Guidance for Industry: Estimating the Maximum Safe Starting Dose in Initial Clinical Trials for Therapeutics in Adult Healthy Volunteers; Nair and Jacob 2016). Accordingly, for a 30 g mouse, 1.5 mg of tryptophan is needed to observe sleep related efficacy. In the current study tested, the level of tryptophan in the chlorella is 1.1 g/100 g (i.e. 1.1%). As such, it is only the highest dosage tested (5000 mg/kg) is expected to provide possible positive outcome for sleep impact (5000 mg/kg×0.03 kg=150 mg chlorella or 1.5 mg tryptophan). However, the addition of Zea mays (corn) leaf extract standardized at 6-MBOA has improved the effect of lower dosages i.e. the 1000 mg/kg and 2000 mg/kg chlorella significantly. This is because of the unique MOA of Zea mays (corn) leaf extract standardized at 6-MBOA which is known to reduce the activity of TDO enzyme to free up more starting material (tryptophan) and enhancing the activity of rate limiting enzyme TH during the biosynthesis of melatonin. In the current preparation of chlorella, even at the highest dosage (which is 5000 mg/day for human usage), the level of tryptophan is only 50 mg/day. This is significantly (five-fold) lower than the expected 250 mg tryptophan level to produce any impact on human physiological sleep (Sutanto et al., 2022). The addition of Zea mays (corn) leaf extract standardized at not less than 0.2% 6-MBOA boosted the efficacy of chlorella by preserving the available tryptophan as starting material and facilitating the biosynthesis of melatonin.

The unexpected synergistic effect of combining Zea mays (corn) leaf extract standardized at 6-MBOA with chlorella in enhancing sleep parameters is also coming from the ability of Zea mays (corn) leaf extract standardized at 6-MBOA increasing the level of tryptophan through the reduction of cortisol. Glucocorticoids such as cortisol are known to induce TDO enzyme leading to hepatic degradation of tryptophan rendering less tryptophan available for melatonin conversion (Altman and Greengard, 1966). Clinically, Zea mays (corn) leaf extract standardized at 6-MBOA has shown significant reduction in the early morning salivary level of cortisol. As such, Zea mays (corn) leaf extract standardized at 6-MBOA can directly inhibit the TDO enzyme and indirectly through the reduction of cortisol with a net result of increased tryptophan availability from chlorella consumption for the biosynthesis of serotonin and melatonin.

A Zea mays (corn) leaf extract standardized at not less than 0.2% 6-MBOA content, has previously shown to improve quality of sleep by improving the deep sleep time, and reducing stress hormone cortisol. Zea mays (corn) leaf extract neither in preclinical in vivo studies nor in human clinical studies showed shortening of sleep onset latency. Although it has never been demonstrated, Chlorella, rich source of nutrients and micronutrients has been implied for its usage in sleep support. As disclosed herein, Chlorella and Zea mays (corn) leaf extract standardized at 6-MBOA were formulated in different ratios and tested in pentobarbital induced sleep study in mice. The combinations of Chlorella and Zea mays (corn) leaf extract standardized at 6-MBOA demonstrated a) statistically significant shortening of sleep onset latency when compared to individual constituents; b) up to 44.4% reductions in the sleep latency standard deviation minimizing the variations in benefits between individuals; c) statistically significant reduction in sleep onset latency when compared to melatonin (used as a positive control compound); and d) unexpected synergistic effect in increasing incidence of falling asleep was observed indicating the synergistic benefit of combining these two materials. The composition of Chlorella and Zea mays (corn) leaf extract standardized at 6-MBOA also increased sleep incidences and total sleep times.

Chlorella is a genus of eukaryotic, single-celled green algae found in freshwater, marine and edaphic habitats (Bock, Krienitz, and Proschold 2011). Identification of species within this genus had historically relied on morphology and nutritional requirements, but more accurate identification using sequencing techniques are preferred (Huss et al., 1999).

C. sorokiniana was first isolated from a creek in Austin, Texas in 1951 (Bock, Krienitz, and Proschold 2011). Although C. vulgaris and C. sorokiniana are morphologically identical, C. sorokiniana can be separated from C. vulgaris by hydrogenase activity, preference for warmer growth temperature, and the sequence of rRNA genes (Huss et al. 1999).

Live C. sorokiniana CK-22 cells are spherical to slightly ovoid, measure 3-6 μm in diameter and have a thin cell wall. C. sorokiniana CK-22 has cup-shaped chloroplasts with a starch grain-covered pyrenoid. These morphological characteristics also correspond with those of C. vulgaris. Although C. sorokiniana CK-22 was originally identified as C. vulgaris CK-22 by morphology, CK-22 has been redesignated as C. sorokiniana following 18S rRNA sequencing.

Herein, Chlorella Powder is the spray-dried whole cell biomass of C. sorokiniana CK-22, as used in contemplated embodiments with its production scale cultivation disclosed in Example 5 and 6 and characterization disclosed in Example 7.

In contemplated embodiments, chlorella serves as a source of tryptophan that led to the benefit in the biosynthesis of melatonin. Chlorella is a type of green algae that is often consumed as a dietary supplement known for its rich nutritional profile, including high levels of protein (Example 7, Table 5). Tryptophan is one of the essential amino acids found in the protein of chlorella (Example 7, Table 6). As an essential amino acid, tryptophan cannot be synthesized by the human body and must be obtained through the diet (such as chlorella supplementation). Tryptophan serves as a precursor for the synthesis of serotonin and melatonin, which play roles in mood regulation and sleep-wake cycles.

As an alternative to conventional drugs, the use of physiological agents as a sleep aid product has been reported. Since a natural metabolic pathway already exists, for these physiological agents to modulate sleep, their use may have a reduced risk of undesirable side effects and toxicity. Tryptophan is one of those physiological agents and it can be obtained from protein-rich food and supplementation such as chlorella. However, the impact of tryptophan on sleep is mixed; one of the major concerns of its efficacy is the large daily dosage needed. Though 250 mg of tryptophan may improve sleep, administration of 1-5 g dose of tryptophan per day had a more consistent favorable effect on sleep (Sutanto et al., 2022).

Once consumed, tryptophan will undergo several chemical reactions before its conversion to serotonin and melatonin. Tryptophan dioxygenase (TDO) is an enzyme that catalyzes the first and rate-limiting step in the kynurenine pathway, which is the major pathway for tryptophan metabolism (Davis and Liu 2015). TDO is primarily found in the liver, where it regulates the levels of tryptophan in the body. The activity of TDO is influenced by factors such as the levels of tryptophan (as in chlorella supplementation increases tryptophan level) and hormones including glucocorticoids such as cortisol (cortisol is known to induce TDO hence reducing cortisol level will increase the level of tryptophan) (Jovanovic et al., 2023). As such, inhibiting the activity of this rate limiting enzyme, will provide surplus tryptophan as the starting material for the biosynthesis of serotonin and melatonin. In fact, Zea mays (corn) leaf extract standardized at not less than 6-MBOA is known to inhibit TDO directly and through reduction of a stress hormone cortisol. These orchestrated effect of Zea mays (corn) leaf extract standardized at 6-MBOA and chlorella is the key mechanism working together for contemplated embodiments that led to shortened sleep onset latency, promotion of sleep and improved sleep quality.

Previously filed and commonly owned US patent application entitled “Composition for regulating homeostasis of cortisol and improving sleep quality and method of use and production thereof, US 2022/0387366 A1” is referred to herein and incorporated by reference. Zea mays (corn) leaf extract standardized at 6-MBOA disclosed in Examples 1 and 2 herein is enriched and standardized for one or more benzoxazinoid 6-MBOA, as contemplated herein. Contemplated benzoxazinoids isolated from corn shoot or immature corn leaf powder are extracted with any suitable solvent, including water, methanol, ethanol, acetone, alcohol, a water-mixed solvent or a combination thereof or with supercritical fluid. In contemplated embodiments, the corn shoot or immature corn leaf extract comprises about 0.01% to about 99.9% benzoxazinoids.

It is contemplated that benzoxazinoids such as 6-MBOA are derived, obtained or selected from other species of plant as demonstrated in Examples 3 and 4 from at least one of the following-alone or in combination with one another seedlings and all plant parts of corn, wheat, rye, rice, barley, oat, cereal, adlay, sorghum plants and other plants such as Zea mays, Oryza species, Oryza sativa, Oryz glaberrima, Oryz australiensis, Oryz brachyantha, Secale cereale, Acanthus arboreus; Acanthus illicifolius, Avena sativa, Avena abyssinica, Avena byzantine, Avena nuda, Avena strigosa, Hordeum vulgare, Coix lachryma-jobi, Triticum aestivum, Triticum compactum, Triticum sphaerococcum, Triticum turanicum, Sorghum bicolor, and Balsamocitrus paniculate; Peristrophe roxburghiana; Strobilanthes cusia; Scoparia dulcis, Lobelia chinensis, Leymus chinensis, a marine sponge Oceanapia sp. or a combination thereof.

The composition enriched for one or more benzoxazoles, is obtained, derived or extracted from any suitable source or sources, including but not limited to seedlings, shoots, germinates from plant seeds, sprouts of geminated grains, immature leaves, mature leaves, whole plants, roots, seeds, flowers, stems, stem barks, root barks, silk, grain, hair roots or rootlets of germinated grain, stem cells, cell cultures or tissue cultures or any combination thereof corn, wheat, rye, rice, barley, oat, cereal, adlay, sorghum plants and other species of plants including but not limited to Zea mays, Oryza species, Oryza sativa, Oryz glaberrima, Oryz australiensis, Oryz brachyantha, Secale cereale, Acanthus arboreus; Acanthus ebracteatus, Acanthus illicifolius, Acanthus mollis, Avena sativa, Avena abyssinica, Avena byzantine, Avena nuda, Avena strigosa, Hordeum vulgare, Coix lachryma-jobi, Triticum aestivum, Triticum compactum, Triticum sphaerococcum, Triticum turanicum, Sorghum bicolor, Agropyron repens, Blepharis edulis, Balsamocitrus paniculate; Peristrophe roxburghiana; Strobilanthes cusia; Lamium galeobdolon, Lobelia chinensis, Leymus chinensis, Aphelandra spp, Scoparia dulcis, Capparis sikkimensis ssp, a fungal species Monocillium sp, a marine sponge Oceanapia sp. or a combination thereof.

Contemplated compositions enriched for one or more benzoxazoles are synthesized, metabolized, biodegraded, bioconverted, biotransformed, biosynthesized from small carbon units, by transgenic microbial, by P450 enzymes, by glycotransferase enzyme or a combination of enzymes, by microbacteria.

Contemplated compositions, wherein one or more benzoxazoles in the compositions in combination with chlorella shortens sleep latency, increase sleep incidence, improves sleep quality by enhancing the deep sleep stage of sleep, increase total sleep time and deep sleep time, improve REM sleep and NREM sleep, improve overall mental well-being measured by the Pittsburgh Sleep Quality Index (PSQI) and Profile of Mood States (POMS), provide positive mood support and enhance emotional well-being; and maintain homeostasis of biomarkers-serotonin, melatonin, GABA in a mammal.

Contemplated compositions, wherein benzoxazoles extracted and standardized from Zea mays leaf are in the compositions in combination with chlorella, prevents and treats sleep disorders including but not limited to insomnia, hypersomnia, jet lag, circadian rhythm disorders, shift work sleep disorder, non-24-hour sleep-wake disorder, periodic limb movement disorder, restless legs syndrome (RLS), sleep apnea, narcolepsy, parasomnias, night terrors, sleepwalking, nightmares, sleep eating disorder. sleep hallucinations, sleep paralysis, sleep talking, and REM Sleep Behavior Disorder.

A contemplated composition, wherein one or more benzoxazoles in the composition in combination with chlorella establishes and regulates homeostasis of host stress hormone, cortisol, and leads to improved symptoms of chronically high cortisol, including but not limited to anxiety, depression, fatigue, gastrointestinal upset like constipation, bloating, or diarrhea, headache, heart disease, high blood pressure, irritability, problems with memory and concentration, reproductive issues like low libido, erectile dysfunction, or irregular menstruation and ovulation, sleep difficulties, slow recovery from exercise, eating disorders and weight gain, hot flashes and other menopausal associated mental and health conditions.

In the above and following descriptions, certain specific details are set forth in order to provide a thorough understanding of various embodiments of this disclosure. However, one skilled in the art will understand that contemplated embodiments may be practiced without these details.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “and/or”) should be understood to mean either one, both, or any combination thereof of the alternatives. Unless the context requires otherwise, throughout the present specification and claims, the word “comprise” and variations thereof, such as, “comprises” and “comprising,” as well as synonymous terms like “include” and “have” and variants thereof, are to be construed in an open, inclusive sense; that is, as “including, but not limited to.”

Reference throughout this specification to “one embodiment” or “an embodiment” means that a particular feature, structure or characteristic described in connection with the embodiment is included in at least one embodiment of contemplated embodiments. Thus, the appearances of the phrases “in one embodiment” or “in an embodiment” in various places throughout this specification are not necessarily all referring to the same embodiment.

“Stable compound” and “stable structure” are meant to indicate a compound that is sufficiently robust to survive isolation to a useful degree of purity from a reaction mixture, and formulation into an efficacious therapeutic agent.

“Biomarker(s)” or “marker(s)” component(s) or compound(s) are meant to indicate one or multiple indigenous chemical component(s) or compound(s) in the disclosed plant(s), plant extract(s), or combined composition(s) with 2-3 plant extracts that are utilized for controlling the quality, consistence, integrity, stability, and/or biological functions of the invented composition(s). Some times the quality marker compound(s) is (are) not bioactive compound(s) that directly related to the intended method of use.

“Mammal” includes humans and both domestic animals, such as laboratory animals or household pets (e.g., cats, dogs, swine, cattle, sheep, goats, horses, rabbits), and non-domestic animals, such as wildlife or the like.

“Optional” or “optionally” means that the subsequently described element, component, event or circumstances may or may not occur, and that the description includes instances where the element, component, event or circumstance occur and instances in which they do not. For example, “optionally substituted aryl” means that the aryl radical may or may not be substituted and that the description includes both substituted aryl radicals and aryl radicals having no substitution.

“Pharmaceutically or nutraceutically or functional food acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically or nutraceutically or functional food acceptable salt” includes both acid and base addition salts. “Pharmaceutically or nutraceutically or functional food acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, ascorbic acid, aspartic acid, cinnamic acid, citric acid, formic acid, fumaric acid, lactic acid, lauric acid, maleic acid, malic acid, malonic acid, mandelic acid, pyruvic acid, salicylic acid, 4-aminosalicylic acid, sebacic acid, stearic acid, succinic acid, tartaric acid, and the like.

“Pharmaceutically or nutraceutically or functional food acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include the sodium, potassium, lithium, ammonium, calcium, magnesium, iron, zinc, copper, manganese, aluminum salts and the like. In certain embodiments, the inorganic salts are ammonium, sodium, potassium, calcium, or magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly useful organic bases are isopropylamine, diethylamine, ethanolamine, trimethylamine, dicyclohexylamine, choline and caffeine.

Often crystallizations produce a solvate of the compound of this disclosure. As used herein, the term “solvate” refers to an aggregate that comprises one or more molecules of a compound of this disclosure with one or more molecules of solvent. The solvent may be water, in which case the solvate may be a hydrate. Alternatively, the solvent may be an organic solvent. Thus, the compounds that are part of contemplated embodiments may exist as a hydrate, including a monohydrate, dihydrate, hemihydrate, sesquihydrate, trihydrate, tetrahydrate and the like, as well as the corresponding solvated forms. The compound of this disclosure may be true solvates, while in other cases, the compound of this disclosure may merely retain adventitious water or be a mixture of water plus some adventitious solvent.

A “pharmaceutical composition” or “nutraceutical composition” or “functional food composition” refers to a formulation of a compound of this disclosure and a medium generally accepted in the art for the delivery of the biologically active compound to mammals, e.g., humans. For example, a pharmaceutical composition of the present disclosure may be formulated or used as a stand alone composition, or as a component in a prescription drug, an over the counter (OTC) medicine, a botanical drug, an herbal medicine, a natural medicine, a homeopathic agent, or any other form of health care product reviewed and approved by a government agency. Exemplary nutraceutical or functional food compositions of the present disclosure may be formulated or used as a stand alone composition, or as a nutritional or bioactive component in food, a functional food, a beverage, a bar, a RTD, a sachet, a food flavor, a medical food, a dietary supplement, or an herbal product. A medium generally accepted in the art includes all pharmaceutically or nutraceutically or functional food acceptable carriers, diluents or excipients therefor.

As used herein, “enriched for” refers to a plant extract or other preparation having at least a two-fold up to about a 1000-fold increase of one or more active compounds as compared to the amount of one or more active compounds found in the weight of the plant material or other source before extraction or other preparation. In certain embodiments, the weight of the plant material or other source before extraction or other preparation may be dry weight, wet weight, or a combination thereof.

As used herein, “major active ingredient” or “major active component” refers to one or more active compounds found in a plant extract or other preparation or enriched for in a plant extract or other preparation, which is capable of at least one biological activity. In certain embodiments, a major active ingredient of an enriched extract will be the one or more active compounds that were enriched in that extract. Generally, one or more major active components will impart, directly or indirectly, most (i.e., greater than 50%, or 20% or 10%, or 1%, or 0.2% or 0.05%, or 0.01%) of one or more measurable biological activities or effects as compared to other extract components. In certain embodiments, a major active ingredient may be a minor component by weight percentage of an extract (e.g., less than 50%, 25%, or 10% or 5% or 1% or 0.2% or 0.05%, or 0.01% of the components contained in an extract) but still provide most of the desired biological activity. Any composition of this disclosure containing a major active ingredient may also contain minor active ingredients that may or may not contribute to the pharmaceutical or nutraceutical activity of the enriched composition, but not to the level of major active components, and minor active components alone may not be effective in the absence of a major active ingredient.

“Effective amount” or “therapeutically effective amount” refers to that amount of a compound, an extract, an algea powder or composition of this disclosure which, when administered to a mammal, such as a human, is sufficient to improve sleep latency, reduce sleep disturbance and fragmentation, improve sleep quality and efficiency through any one or combination of pathways such as 1) reduced nocturnal and/or diurnal plasma, urine or salivary cortisol level, 2) bind to melatonin receptors and increased melatonin synthesis, 3) modulation of hypothalamus-pituitary-adrenal axis, 4) impact on non-rapid eye movement stages of sleep such as slow wave deep sleep, 5) activity on rapid eye movement stage of sleep, and 6) decreasing night time wakefulness.

The amount of a compound, an extract or a composition of this disclosure that constitutes a “therapeutically effective amount” will vary depending on the bioactive compound, or a standardized extract, or an ethanol extract or the biomarker for the condition being treated and its severity, the manner of administration, the duration of treatment, or the age of the subject to be treated, but can be determined routinely by one of ordinary skill in the art having regard to his own knowledge and to this disclosure. In certain embodiments, “effective amount” or “therapeutically effective amount” may be demonstrated as the quantity of bioactive compound or an extract over the body weight of a mammal (i.e., 0.001 mg/kg, 0.005 mg/kg, 0.01 mg/kg, or 0.1 mg/kg, or 1 mg/kg, or 5 mg/kg, or 10 mg/kg, or 20 mg/kg, or 50 mg/kg, or 100 mg/kg, or 200 mg/kg or 500 mg/kg or 1,000 mg/kg, or 2,000 mg/kg or 5,000 mg/kg). The human equivalent daily dosage can be extrapolated from the “effective amount” or “therapeutically effective amount” in an animal study by utilization of FDA guideline in consideration the difference of total body areas and body weights of animals and human.

“Dietary supplements” or “functional foods” as used herein are a product that improves, promotes, increases, manages, controls, maintains, optimizes, modifies, reduces, inhibits, balance, a particular condition associated with a natural state or biological process, or a structural and functional integrity, an off-balanced or a compromised, or suppressed or impaired or overstimulated of a biological function or a phenotypic condition (i.e., are not used to diagnose, treat, mitigate, cure, or prevent disease). For example, with regard to sleep, dietary supplements or functional foods may be used to modulate, maintain, manage, balance, suppress or stimulate any components of sleep and neuroendocrine system to yield a reduced sleep latency, an increased deep sleep time or total sleep time, an improved REM sleep or NREM sleep, an improved sleep quality, efficiency, and duration by correcting factors that would contribute to sleep disturbance, fragmentation, wakefulness, hyperactivity and/or disturbed HPA axis and increased serotonin and melatonin level and reduced cortisol level. In certain embodiments, dietary supplements and functional foods are a special category of food, beverage, RTD, food bar, sachet, powder, tincture, gummy, medical food and are not a drug.

“Treating” or “treatment” as used herein refers to the treatment of the disease or condition of interest in a mammal, such as a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving or modifying the disease or condition, i.e., causing regression of the disease or condition; or (iv) relieving the symptoms resulting from the disease or condition, (e.g., reducing sleep latency, improving sleep quality and efficiency of patient diagnosed with a sleep disorder such as insomnia) without addressing the underlying disease or condition; (v) balancing the regulation of HPA axis homeostasis or changing the phenotype of the disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians. A disease or condition may be acute such as insomnia; and may be chronic such as sleep disorder caused by aging. A compromised hypothalamic-pituitary-adrenal (HPA) axis function from off balance of homeostasis could cause a disease or a condition, or could make the Mammal more susceptible neurological disorders, or could lead to more acutely or chronically elevated cortisol directly or indirectly associated with sleep disorders.

As used herein, “statistical significance” refers to a p value of 0.05 or less when calculated using the Students t-test and indicates that it is unlikely that a particular event or result being measured has arisen by chance.

For the purposes of administration, contemplated compositions and compounds may be administered as a raw ingredient or may be formulated as pharmaceutical or nutraceutical compositions. Contemplated pharmaceutical or nutraceutical compositions comprise a compound of structures as described and contemplated herein and a pharmaceutically or nutraceutically acceptable carrier, diluent or excipient. The compound of structures described here are present in the composition in an amount which is effective to treat a particular disease or condition of interest—that is, in an amount sufficient to promote good sleep quality and efficiency as well as HPA axis homeostasis in general or any of the other associated indications described herein, and generally with acceptable toxicity to a patient.

Administration of the compounds or compositions of this disclosure, or their pharmaceutically or nutraceutically acceptable salts, in pure form or in an appropriate pharmaceutical or nutraceutical composition, can be carried out via any of the accepted modes of administration of agents for serving similar utilities. The pharmaceutical or nutraceutical or functional food compositions of this disclosure can be prepared by combining the composition of this disclosure with an appropriate pharmaceutically or nutraceutically or functional food acceptable carrier, diluent or excipient, and may be formulated into preparations in solid, semi solid, liquid or gaseous forms, such as tablets, capsules, powders, gummy, granules, ointments, solutions, beverage, suppositories, injections, inhalants, gels, creams, lotions, tinctures, sachet, ready to drink, masks, microspheres, and aerosols. Typical routes of administering such pharmaceutical or nutraceutical compositions include oral, topical, transdermal, inhalation, parenteral, sublingual, buccal, rectal, vaginal, or intranasal. The term parenteral as used herein includes subcutaneous injections, intravenous, intramuscular, intrasternal injection or infusion techniques.

Pharmaceutical or nutraceutical compositions of this disclosure are formulated so as to allow the active ingredients contained therein to be bioavailable upon administration of the composition to a patient. Compositions that will be administered to a subject or patient or a mammal take the form of one or more dosage units, where for example, a tablet may be a single dosage unit, and a container of a compound or an extract or a composition of 2-3 plant extracts of this disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of this disclosure, or a pharmaceutically or nutraceutically acceptable salt thereof, for treatment of a disease or condition of interest in accordance with the teachings of this subject matter.

A pharmaceutical or nutraceutical composition of this disclosure may be in the form of a solid or liquid. In one aspect, the carrier(s) are particulate, so that the compositions are, for example, in tablet or in powder form. The carrier(s) may be liquid, with the compositions being, for example, oral syrup, injectable liquid or an aerosol, which is useful in, for example, inhalatory administration.

When intended for oral administration, the pharmaceutical or nutraceutical composition is in either solid or liquid form, where semi solid, semi liquid, suspension and gel forms are included within the forms considered herein as either solid or liquid.

As a solid composition for oral administration, the pharmaceutical or nutraceutical composition may be formulated into a powder, granule, compressed tablet, pill, capsule, gummy, chewing gum, sachet, wafer, bar, or like form. Such a solid composition will typically contain one or more inert diluents or edible carriers. In addition, one or more of the following may be present: binders such as carboxymethylcellulose, ethyl cellulose, cyclodextrin, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical or nutraceutical composition is in the form of a capsule, for example, a gelatin capsule, it may contain, in addition to materials of the above type, a liquid carrier such as polyethylene glycol or oil.

The pharmaceutical or nutraceutical composition may be in the form of a liquid, for example, an elixir, tincture, syrup, solution, emulsion or suspension. The liquid may be for oral administration or for delivery by injection, as two examples. When intended for oral administration, a useful composition contains, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical or nutraceutical or functional food compositions of this disclosure, whether they be solutions, suspensions or other like form, may include one or more of the following adjuvants: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a generally useful adjuvant. An injectable pharmaceutical or nutraceutical composition is sterile.

A liquid pharmaceutical or nutraceutical composition of this disclosure intended for either parenteral or oral administration should contain an amount of a compound of this disclosure such that a suitable dosage will be obtained.

The pharmaceutical or nutraceutical composition of this disclosure may be intended for topical administration, in which case the carrier may suitably comprise a solution, emulsion, cream, lotion, ointment, or gel base or a patch. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical or nutraceutical composition for topical administration. If intended for transdermal administration, the composition may include a transdermal patch or iontophoresis device.

The pharmaceutical or nutraceutical composition of this disclosure may be intended for rectal administration, in the form, for example, of a suppository, which will melt in the rectum and release the drug. The composition for rectal administration may contain an oleaginous base as a suitable nonirritating excipient. Such bases include lanolin, cocoa butter and polyethylene glycol.

The pharmaceutical or nutraceutical composition of this disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical or nutraceutical composition of this disclosure in solid or liquid form may include an agent that binds to the compound of this disclosure and thereby assists in the delivery of the compound. Suitable agents that may act in this capacity include a monoclonal or polyclonal antibody, a protein or a liposome.

The pharmaceutical or nutraceutical composition of this disclosure in solid or liquid form may include reducing the size of a particle to, for example, improve bioavailability. The size of a powder, granule, particle, microsphere, or the like in a composition, with or without an excipient, can be macro (e.g., visible to the eye or at least 100 μm in size), micro (e.g., may range from about 100 μm to about 100 nm in size), nano (e.g., may no more than 100 nm in size), and any size in between or any combination thereof to improve size and bulk density.

The pharmaceutical or nutraceutical composition of this disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of this disclosure may be delivered in single phase, bi phasic, or tri phasic systems in order to deliver the active ingredient(s). Delivery of the aerosol includes the necessary container, activators, valves, sub-containers, and the like, which together may form a kit. One skilled in the art, without undue experimentation, may determine the most appropriate aerosol(s).

The pharmaceutical or nutraceutical compositions of this disclosure may be prepared by methodology well known in the pharmaceutical or nutraceutical art. For example, a pharmaceutical or nutraceutical composition intended to be administered by injection can be prepared by combining a compound of this disclosure with sterile, distilled, deionized water so as to form a solution. A surfactant may be added to facilitate the formation of a homogeneous solution or suspension. Surfactants are compounds that non covalently interact with the compound of this disclosure so as to facilitate dissolution or homogeneous suspension of the compound in the aqueous delivery system.

The compounds of this disclosure, or their pharmaceutically or nutraceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of this disclosure, or pharmaceutically or nutraceutically acceptable derivatives thereof, may also be administered simultaneously with, prior to, or after administration of food, water and one or more other therapeutic agents. Such combination therapy includes administration of a single pharmaceutical or nutraceutical dosage formulation which contains a compound or an extract or a composition with 2-3 plant extracts of this disclosure and one or more additional active agents, as well as administration of the compound or an extract or a composition with 2-3 plant extracts of this disclosure and each active agent in its own separate pharmaceutical or nutraceutical dosage formulation. For example, a compound or an extract or a composition with 2-3 plant extracts of this disclosure and another active agent can be administered to the patient together in a single oral dosage composition, such as a tablet or capsule, or each agent can be administered in separate oral dosage formulations. Where separate dosage formulations are used, the compounds of this disclosure and one or more additional active agents can be administered at essentially the same time, i.e., concurrently, or at separately staggered times, i.e., sequentially; combination therapy is understood to include all these regimens.

It is understood that in the present description, combinations of substituents or variables of the depicted formulae are permissible only if such contributions result in stable compounds.

It will also be appreciated by those skilled in the art, although such protected derivatives of compounds included as contemplated embodiments may not possess pharmacological activity as such, they may be administered to a mammal and thereafter metabolized in the body to form compounds of this disclosure which are pharmacologically active. Such derivatives may therefore be described as “prodrugs”. All prodrugs of compounds included as contemplated embodiments are included within the scope of this disclosure.

Furthermore, all compounds or extracts of this disclosure which exist in free base or acid form can be converted to their pharmaceutically or nutraceutically acceptable salts by treatment with the appropriate inorganic or organic base or acid by methods known to one skilled in the art. Salts of the compounds of this disclosure can be converted to their free base or acid form by standard techniques.

Contemplated compounds, medicinal compositions and compositions may comprise or additionally comprise or consist of at least one active ingredient. In some embodiments, at least one bioactive ingredient may comprise or consist of plant powder or plant extract of or the like.

In any of the aforementioned embodiments, the standardized extract comprising mixtures of water or alcohol or supercritical fluid extracts of a composition derived from enriched for one or more benzoxazoles is mixed with chlorella at a particular ratio by weight. In certain embodiments, the ratio (by weight) of at least one or more benzoxazole extract mixed with Chlorella in ranges from about 0.05:99.95 to about 99.95:0.05. Similar ranges apply when more than two extracts or compounds (e.g., three, four, five) are used. Exemplary ratios of benzoxazole extract: Chlorella include but not limited to 0.05:99.95, 0.1:99.9, 0.15:99.85, 0.2:99.8, 0.25:99.75, 0.3:99.7, 0.4:99.6, 0.5:99.5, 0.6:99.4, 1:99, 2:98, 3:97, 4:96, 5:95, 6:94, 7:93, 8:92, 9:91, 10:90, 15:85, 20:80, 25:75, 30:70, 40:60, 50:50, 60:40, 70:30, 80:20, 90:10, 95:5, 99:1, 99.5:0.5, 99.9:0.1, 99.95:0.05. In further embodiments, the disclosed standardized extract of a composition derived from enriched for one or more benzoxazoles coded as UP165 from Zea mays leaves in combination with Chlorella has been combined into a composition as an example but not limited to be enriched to or standardized at 0.2% of a quality marker compound: 6-MBOA. In further embodiments, such enrichment or standardization of Zea mays extract is mixed one or more benzoxazoles either naturally isolated or artificially synthesized with equivalent chemical structure(s) of natural compound(s) and combined with Chlorella.

In further embodiments, such enrichment or standardization of Zea mays extract in combination with chlorella, were evaluated on in vitro, and/or ex vivo and/or in vivo models for advantage/disadvantage and unexpected synergy/antagonism of the perceived biological functions such as shortening of sleep onset latency, sleep incidence, total sleep time, deep sleep time,

RAM sleep time, NREM sleep time, and effective adjustments of the homeostasis of neuroendocrine function, increases of Serotonin, Melatonin, GABA, DHEA and reduction of cortisol to yield improved sleep quality, improved mood status in human clinical trial. The best standardization with specific blending ratio of individual compounds were selected based on unexpected synergy measured on the in vitro, and/or ex vivo and/or in vivo models and potential enhancement of ADME of these compounds maximize the biological outputs.

In certain examples, a composition of this disclosure may be formulated to further comprise a pharmaceutically or nutraceutically acceptable carrier, diluent, or excipient, wherein the pharmaceutical or nutraceutical formulation comprises from about 0.01 or 0.05 weight percent (wt %), or or 0.2% or, 0.5 weight percent (wt %), or 5%, or 25% to about 95 wt % of active or major active, or biomarker compound(s) of an extract mixture. In any of the aforementioned formulations, a composition of this disclosure is formulated as a tablet, hard capsule, soft gel capsule, powder, gummy, RTD or granule.

Also contemplated herein are agents of the disclosed compounds. Such products may result from, for example, the oxidation, reduction, hydrolysis, amidation, esterification, and the like of the administered compound, primarily because of enzymatic processes. Accordingly, contemplated compounds are those produced by a process comprising administering a contemplated compound or composition to a mammal for a period of time sufficient to yield a metabolic product thereof. Such products are typically identified by administering a radiolabeled or not radiolabeled compound of this disclosure in a detectable dose to an animal, such as rat, mouse, guinea pig, dog, cat, pig, sheep, horse, monkey, or human, allowing sufficient time for metabolism to occur, and then isolating its conversion products from the urine, blood or other biological samples.

Contemplated compounds, medicinal compositions and compositions may comprise or additionally comprise or consist of at least one pharmaceutically or nutraceutically or cosmetically acceptable carrier, diluent or excipient. As used herein, the phrase “pharmaceutically or nutraceutically or cosmetically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. Contemplated compounds, medicinal compositions and compositions may comprise or additionally comprise or consist of at least one pharmaceutically or nutraceutically or cosmetically acceptable salt. As used herein, the phrase “pharmaceutically or nutraceutically or cosmetically acceptable salt” includes both acid addition and base addition salts.

In any of the aforementioned embodiments, the compositions comprising the enriched or standardized corn leaf extract, in combination with chlorella and combination of one or more bioactive extracts or compounds to complement or boost the effects for shorting of sleep onset latency, regulating homeostasis of cortisol and improving sleep quality that may be present at certain percentage levels or ratios. In certain contemplated embodiments, compositions can include a composition that contain Chlorella and corn leaf extract enriched for one or more benzoxazoles or standardized corn leaf extract as one of biomarker compounds and/or extracts from about 0.01% to about 99.9% benzoxazoles and/or its like compounds, or derivatives or precursors that can be isolated from natural sources or synthesized.

Natural bioactive compounds, Chlorella or extracts in combination with enriched or standardized benzoxazoles in Zea mays (corn) leaf or corn shoot extract disclosed in current contemplated embodiments contain molecules that shorten sleep onset latency, enhance biosynthesis of serotonin and melatonin, modulate the HPA axis and normalize the cortisol level for a homeostatic feedback that will lead to an improved sleep quality and efficiency. Those natural compounds to be combined further with current contemplated embodiments disclosed compositions containing benzoxazoles in nutraceutical or functional food products include but not limited to Melatonin, Magnesium, gamma aminobutyric acid (GABA), CBD, vitamin B1, B2, B3, B6, B12, Pyridoxine, Methylcobalamin, Niacinamide, Folic acid, Ascorbic acid, vitamin C, Vitamin D & E, Zinc, omega-3 fatty acid, Glycine, Glutamine, arginine, Tryptophan, L-theanine, 5-Hydroxytryptophan (5-HTP), SAMe, Magnolol, Honokiol, Taurine, Boron, Branched-Chain amino acids (BCAA), phospholipids, phosphatidylserine, phosphatidic acid, theaflavin, rosmarinic acid, catechin, epicatechin, conjugated catechins such as EGCG, ECG, epigallocatechin etc. baicalein, baicalin, Oroxylin, Wogonin, Kaempferol, genistein, quercetin, Butein, Betaine, Luteolin, chrysin, Apigenin, curcumin, resveratrol, glomeratose A, 6-shogaol, gingerol, berberine, piperine, carnosol and carnosic acids.

The plant species that can be in combination with contemplated embodiments containing Chlorella and benzoxazoles standardized Zea mays leaf extract in nutraceutical and functional food products to shorten sleep onset latency, enhance biosynthesis of serotonin and melatonin, modulate the HPA axis and normalize the cortisol level for a homeostatic feedback that will lead to a reduced sleep latency, an improved sleep quality and efficiency including but not limited to Valerian roots, Valeriana officinalis, Ginkgo biloba, Kava kava, Lavender, Passionflower (Passiflora incarnata or maypop), Chamomile flower, Hops, Humulus lupulus, Hibiscus sabdariffa, St. John's Worth, Griffonia simplicifolia, Fermented milks, fish oil, Rhodiola rosea, Lotus seed, Lotus seed germ, Oryza sativa, Zea mays, Ziziphus jujuba, Schisandra chinensis, Magnolia officinalis, Astragalus membranaceus, Ganoderma lucidum, Echinacea purpurea, Echinacea angustifolia, Poria cocos Wolf, Wolfiporia extensa, Ashwagandha, Withania somnifera, Crocus sativus (Saffron), Bupleurum falcatum, Glycyrrhiza spp, Panax quinquefolium, Panax ginseng C. A. Meyer, Korea red ginseng, Eurycoma longifolia (Malaysian ginseng) Lentinula edodes (shiitake), Inonotus obliquus (Chaga mushroom).

The above plant species that can be in combination with contemplated embodiments containing Chlorella and benzoxazoles standardized Zea mays leaf extract in nutraceutical and functional food products can be isolated from plant, and/or marine sources included in the Examples and elsewhere throughout the present application. Suitable plant parts for the extraction and combination with contemplated embodiments containing Chlorella and benzoxazoles standardized Zea mays leaf extract include shoots, sprouts, leaves, immature leaf, bark, trunk, trunk bark, stem, stem bark, twigs, tubers, root, rootlet, rhizome, root bark, bark surface, young shoots, seeds, fruits, seedlings, root hairs, androecium, gynoecium, calyx, stamen, petal, sepal, carpel (pistil), flowers, or any combination thereof. In some related embodiments, the above plant species that can be in combination with contemplated embodiments containing Chlorella and benzoxazoles standardized Zea mays leaf extract in nutraceutical and functional food products can be extracted from plant sources and synthetically made or modified to contain any of the recited substituents. In this regard, synthetic modification of the compound isolated from plants can be accomplished using any number of techniques including biosynthesis, bioengineering, fermentation, enzymatic transformation which are known in the art and are well within the knowledge of one of ordinary skill in the art.

### EXAMPLES

### Example 1

### Preparation of Organic Extracts from Corn Leaves (Zea mays)

Dried ground immature corn leaf powder (Zea mays) (10 g) loaded into two 100 ml stainless steel tube and extracted twice with different organic solvents, including dichloromethane, methanol, ethanol, acetone, petroleum and ethyl acetate using an ASE 300 automatic extractor at 80° C. and pressure 1500 psi. The extract solution is automatically filtered and collected. The organic extract solution is evaporated with rotary evaporator to give crude organic extracts as listed in the table 1.

TABLE 1
Extraction yield of corn leaf extracts

| Solvent | DCM | MeOH | EtOH | Acetone | EtOAc |
|---|---|---|---|---|---|
| Extraction yield (%) | 2.9% | 9.6% | 6.1% | 2.8% | 2.4% |

A composition derived from enriched for one or more phenylpropanoid acids and benzoxazinoids (UP165) was produced as 70% Ethanol/30% water extract of ground immature corn leaf powder at 70-90° C. and standardized with no less than 0.2% 6-MBOA that is isolated from natural sources or synthesized.

Similar results were obtained using the same procedure, but with the organic solvent being replaced with methanol or ethanol to provide a methanol extract (ME) or ethanol extract (EE), methanol:H₂O (7:3) extracts, methanol:H₂O (1:1) extracts, methanol:H₂O (3:7) ethanol:H₂O (7:3) extracts, ethanol:H₂O (1:1) extracts, ethanol:H₂O (3:7) extracts and water extracts respectively.

### Example 2

### 6-MBOA Quantification in Corn Leaf Extracts

The immature corn (Zea mays) leaf extracts (10 mg/mL) were analyzed on a Hitachi HPLC/PDA system with a C18 reversed-phase column (Phenomenex, Luna 5 μm, 150 mm×4.6 mm), eluted with 0.2% formic acid in H₂O and acetonitrile solvent system at a flow rate of 1 mL/min with UV detection at a wavelength of 286 nm with an injection volume of 10 μL against pure 6-MBOA (543551, Sigma-Aldrich) as external reference standard, prepared at a concentration of 0.2 mg/mL with the same injection volume. The 6-MBOA contents in plant extracts were determined in a range of 0.09-0.3% in the extracts obtained by different solvents including but not limited to methanol, ethanol, dichloromethane (DCM), acetone, ethyl acetate.

TABLE 2
6-MBOA content in corn leaf extracts

| | A composition | | | | | |
|---|---|---|---|---|---|---|
| | derived from | | | | | |
| | enriched | | | | | |
| | benzoxazinoids | | | | | |
| Extract | UP165 | DCM | MeOH | EtOH | Acetone | EtOAc |
| 6-MBOA content (%) | 0.2% | 0.22% | 0.09% | 0.15% | 0.13% | 0.3% |

### Example 3

### Preparation of Organic Extract from Scoparia dulcis

Dried ground Scoparia dulcis whole plant powder (20 g) was loaded into 100 ml stainless steel tube and extracted twice with an organic solvent mixture (methylene chloride/methanol in a ratio of 1:1) using an ASE 300 automatic extractor at 80° C. and pressure 1500 psi. The extract solution is automatically filtered and collected. The organic extract solution is evaporated with rotary evaporator to give crude organic extracts (OE, 2.64 g, 13.2%) with 6-MBOA content quantified at 0.58% by HPLC method described in Example 2.

### Example 4

### Evaluation 6-MBOA Contents and Composition Profiles with Enriched for One or More Benzoxazinoids in Different Germinated Crops

Plant seeds of 6 crop species were planted in the prepared soil to grow the plant under the standard agriculture practice grown in Texas in early Spring and whole plant shoots were harvested 10 days after germination. The ground and dried plant shoot powder was extracted with methanol to give methanol extract for each plant. Extracts were prepared at 10 mg/mL concentration and analyzed by ACQUITY UPLC-I-class Xevo G2-XS-QTof system for composition profiles with enriched for one or more phenylpropanoid acids and benzoxazinoids. 6-MBOA was also quantified at 0.66% in corn shoot extract and 0.12% in the corn shoot powder; while 0.19% in wheat shoot extract and 0.03% in the wheatgrass powder; 0.0081% in rye shoot extract and 0.0018% in the rye grass powder. 6-MBOA was not detected in barley, oats, and buckwheat extracts in this study.

The ground and dried corn young shoot and wheatgrass powder were also extracted with water and water with 2% acetic acid. Extracts were prepared at 10 mg/mL concentration and analyzed by ACQUITY UPLC-I-class Xevo G2-XS-QTof system to determine the profiles of enriched one or more phenylpropanoid acids and benzoxazinoids as well as the content for 6-MBOA.

Coix lacryma-jobi L seed was directly extracted with methanol and analyzed by ACQUITY UPLC-I-class Xevo G2-XS-QTof system at a concentration of 50 mg/ml concentration. The profiles of enriched one or more phenylpropanoid acids and benzoxazinoids as well as the 6-MBOA was determined at 0.00226% in the coix seed powder.

TABLE 3
6-MBOA contents in shoot extracts of different plants 10-days after gemination

| Sample | | Common | | 6-MBOA in | 6-MBOA in |
|---|---|---|---|---|---|
| Code | Plant species | name | Extract ID | extract | plant |
| L0879 | Zea mays | Corn | L0879-ME | 0.66% | 0.12% |
| L0880 | Hordeum vulgare | Barley | L0880-ME | Not | Not |
| | | | | detected | detected |
| L0881 | Avena sativa | Oats | L0881-ME | Not | Not |
| | | | | detected | detected |
| L0882 | Triticum aestivum | Wheat | L0882-ME | 0.19% | 0.03% |
| L0883 | Secale cereale | Rye | L0883-ME | 0.0081% | 0.0018% |
| L0884 | Fagopyrum esculentum | Buckwheat | L0884-ME | Not | Not |
| | | | | detected | detected |

TABLE 4
6-MBOA contents in corn and wheat extracts

| | Sample | | Extraction | 6-MBOA in |
|---|---|---|---|---|
| Sample code | description | Extract (g) | Yield | extract |
| L0879-ME | Methanol extract | 3.6849 | 18.42% | 0.66% |
| L0879-AE | Water extract | 3.8667 | 19.33% | 0.41% |
| L0879-AEA | 2% acetic acid | 2.86841 | 14.34% | 0.48% |
| | extract | | | |
| L0882-ME | Methanol extract | 3.2152 | 16.08% | 0.19% |
| L0882-AE | Water extract | 4.4838 | 22.42% | 0.11% |
| L0882-AEA | 2% acetic acid | 4.0747 | 20.37% | 0.07% |
| | extract | | | |

### Example 5

### Ck-22 Culture of Chlorella

CK-22 is cultured and sequentially expanded in the following steps: slant culture, flask culture, jar culture, seed culture, tank culture, and finally, outdoor pool culture.

### 1) Slant Culture

A stock of CK-22 is maintained in slant culture. The slant culture is initiated from the original frozen stock strain once a month. An agar slant culture is inoculated using a sterile loop. This slant culture is managed by successive subculture at Chlorella Industry Co., Ltd. The slant culture is considered successful if the CK-22 culture is dark green in color and proliferating within 1-2 weeks under constant fluorescent light. The slant culture is then used to inoculate the flask culture.

### 2) Flask Culture

All culture vessels and culture medium for the indoor culture steps are sterilized prior to inoculation at each step. The flask culture is inoculated under sterile conditions and cultured under constant mechanical agitation for 1-2 weeks in the presence of glucose with constant exposure to a light source. A portion of the flask culture is then expanded to the jar culture. The remaining culture is discarded.

### 3) Jar Culture

The jar culture is grown with agitation and ambient light. A transport line for seed culture inoculation is connected to the jar culture and the entire jar culture biomass is transferred when cell growth plateaus.

### 4) Seed Culture

After inoculation from the jar culture, the CK-22 culture is grown with constant agitation, and under controlled temperature and pH. The seed culture is grown in the presence of glucose and in the absence of light. A portion of the seed culture is collected and used for the next step, tank culture.

### 5) Tank Culture

A portion of seed culture is transferred to a tank culture vessel containing sterile medium with constant agitation, in the presence of glucose, and the absence of light. Temperature and pH are controlled during tank culture. Once the biomass reaches appropriate chlorophyll content, the entire tank culture is moved to the outdoor pool culture via pipeline.

### 6) Outdoor Pool Culture

The tank culture is then expanded to a shallow, outdoor pool by pipeline and cultured in the presence of ambient light and temperature with agitation. The nitrogen concentration and pH of the culture medium is monitored during the outdoor pool culture as a functional readout of the health of the culture. Accordingly, agitation speed may be increased to maintain the health of the culture. The culture is also monitored through microscopic examination of cell shape, lack of aggregation and to monitor for potential microbial contamination. In the event of a microbial contamination, the culture is discarded. After the appropriate chlorophyll content is reached, the cultured is transferred via a pipeline to be harvested for processing.

### Example 6

### Processing and Production of Chlorella Powder

Processing and production of Chlorella Powder from the outdoor culture includes washing, filtering, and sterilizing the algae.

After the culture process is complete as shown in the example 5, the CK-22 biomass is collected by filtering the culture through 800 μm and 550 μm filters. The culture is then washed and concentrated by three separate centrifugation steps to remove the culture medium. Each wash is done with sterile water, and the culture is passed through a final 350 μm filter. The final slurry is passed through a magnetic strainer, cooled to 2-5° C. and stored for up to 24 hours before undergoing heat inactivation for 3 minutes at 100° C.

The heat sterilized CK-22 is then spray dried. The spray dried intermediate product is sieved through a magnetic strainer, passed through a 20-mesh strainer and then packaged into 100 kg bags. One batch of seed culture corresponds to one lot of intermediate product.

The final product consists of multiple lots of intermediate product that are mixed to yield one 1000-1500 kg finished lot. After mixing, the product goes through a magnetic strainer. The particle distribution of the Chlorella Powder is: 19 μm (10 percentile), 60 μm (50 percentile), 134 μm (90 percentile).

### Example 7

### Specification of Chlorella CK-22 Powder

The characterization of Chlorella described in the example 6 was summarized in the Table 5 and Table 6.

TABLE 5
Specification of chlorella CK-22 powder

| Parameter | Method | LOD | Specification | Lot. 170725 | Lot. 180926 | Lot. 190805 |
|---|---|---|---|---|---|---|
| Appearance | Visual Inspection | — | Green | Green | Green | Green |
| | | | Powder | Powder | Powder | Powder |
| Foreign body | Visual Inspection | — | Not | Not | Not | Not |
| | | | observed | observed | observed | observed |
| Moisture (%) | AOAC 930.15 | — | <7.0 | 5.1 | 5.0 | 5.2 |
| Ash (%) | AOAC 942.05 | — | <11.0 | 7.0 | 7.2 | 7.1 |
| Chlorophyll (%) | Alkaline-pyridine | — | ≥1.5 | 3.0 | 2.5 | 2.8 |
| | methoda | | | | | |
| Chlorophyll b | TLCb | 0.08 g/100 g | Detectable | Detectable | Detectable | Detectable |
| Vitamin B2 | AOAC 970.65 | — | ≥4.0 | 5.9 | 5.7 | 5.9 |
| (mg/100 g) | | | | | | |
| Protein (%) | AOAC 984.13 | — | ≥55.0 | 64.4 | 63.1 | 65.0 |
| Iron (mg/100 g) | AOAC 999.10 | — | 40-100 | 57 | 60 | 63 |
| Fat (%) | AOAC 954.02 | — | ≥8.8 | 13.1 | 11.4 | 11.5 |
| Carbohydrate | Calculated | — | ≥7.0 | 10.4 | 13.5 | 11.2 |
| (%) | | | | | | |
| Omega 3 Fatty | AOAC 991.39 | — | ≥1.00 | 1.64 | 1.39 | 1.67 |
| Acids (%) | | | | | | |
| Vitamin B2 | AOAC 970.65 | — | ≥4.0 | 5.9 | 5.7 | 5.9 |
| (mg/100 g) | | | | | | |
| Protein (%) | AOAC 984.13 | — | ≥55.0 | 64.4 | 63.1 | 65.0 |
| Iron (mg/100 g) | AOAC 999.10 | — | 40-100 | 57 | 60 | 63 |
| Fat (%) | AOAC 954.02 | — | ≥8.8 | 13.1 | 11.4 | 11.5 |
| Carbohydrate | Calculated | — | ≥7.0 | 10.4 | 13.5 | 11.2 |
| (%) | | | | | | |
| Omega 3 Fatty | AOAC 991.39 | — | ≥1.00 | 1.64 | 1.39 | 1.67 |
| Acids (%) | | | | | | |
| Arsenic (ppm) | AOAC 999.10 | 0.5 ppm | <1.0 | N.D. | N.D. | N.D. |
| Lead (ppm) | AOAC 999.10 | 0.2 ppm | <1.0 | N.D. | N.D. | N.D. |
| Cadmium (ppm) | AOAC 999.10 | 0.02 ppm | <0.20 | N.D. | N.D. | N.D. |
| Mercury (ppm) | AOAC 971.21 | 0.01 ppm | <0.10 | N.D. | N.D. | N.D. |
| Chromium (ppm) | USP<730> | 0.5 ppm | <2.0 | N.D. | N.D. | N.D. |
| Aerobic plate | AOAC 966.23 | — | <1000 | 100 | 100 | 100 |
| count (cfu/g) | | | | | | |
| Coliforms | Deoxycholate | — | Negative/1 g | Negative | Negative | Negative |
| | Agar Methodc | | | | | |
| E. coli | USP<62> | — | Negative/1 g | Negative | Negative | Negative |
| Staphylococcus | USP<62> | — | Negative/1 g | Negative | Negative | Negative |
| Salmonella | USP<62> | — | Negative/10 g | Negative | Negative | Negative |
| Pseudomonas | USP<62> | — | Negative/1 g | Negative | Negative | Negative |
| aeruginosa | | | | | | |
| Mold (cfu/g) | USP<62> | — | <100 | <100 | <100 | <100 |
| Yeast (cfu/g) | USP<62> | — | <100 | <100 | <100 | <100 |

TABLE 6
Amino acid of chlorella powder is analyzed by JFRL
(52-1 Motoyoyogi-cho Shibuya-ku Tokyo Japan).

| Items | Results | Methods |
|---|---|---|
| Arginine | 3.68 g/100 g | Automatic amino acid analyzer |
| Lysine | 4.65 g/100 g | Automatic amino acid analyzer |
| Histidine | 1.15 g/100 g | Automatic amino acid analyzer |
| Phenylalanine | 2.74 g/100 g | Automatic amino acid analyzer |
| Tyrosine | 1.97 g/100 g | Automatic amino acid analyzer |
| Leucine | 5.08 g/100 g | Automatic amino acid analyzer |
| Isoleucine | 2.20 g/100 g | Automatic amino acid analyzer |
| Methionine | 1.26 g/100 g | Automatic amino acid analyzer |
| Valine | 3.34 g/100 g | Automatic amino acid analyzer |
| Alanine | 4.61 g/100 g | Automatic amino acid analyzer |
| Glycine | 3.34 g/100 g | Automatic amino acid analyzer |
| Proline | 2.69 g/100 g | Automatic amino acid analyzer |
| Glutamic acid | 6.75 g/100 g | Automatic amino acid analyzer |
| Serine | 2.34 g/100 g | Automatic amino acid analyzer |
| Threonine | 2.63 g/100 g | Automatic amino acid analyzer |
| Aspartic acid | 5.03 g/100 g | Automatic amino acid analyzer |
| Tryptophan | 1.10 g/100 g | HPLC |
| Cystine | 0.80 g/100 g | Automatic amino acid analyzer |
| Amino acid score | 91 | calculation * |
| Total nitrogen | 10.5 g/100 g | combustion method |

### Example 8

### Experimental Design Employed to Evaluate the Synergy of Zea mays (Corn) Leaf Extract Standardized at 6-MBOA and Chlorella

Purpose bred BALB/C mice (n=405; male, 20 g-22 g) were purchased from Beijing Hua Fukang biological technologies Inc (Beijing, China). The study was carried out at Testing Center for the functions of Health Foods, College of Arts and science of Beijing Union University (Beijing, China). Mice were kept in specific pathogen free, temperature-controlled room and fed with rodent diet purchased from Beijing Hua Fukang biological technologies Inc (Beijing, China). Three consecutive studies were carried out. Mice were randomly assigned into 9 groups for each experiment. Fifteen mice were allocated in each group. The first experiment was set to determine the sleep incidence effect of test materials. The second experiment was designed to detect the total sleep time under the hypnotic subthreshold dose of pentobarbital sodium (Lot #: 34E10320, Beijing Dingguo Changsheng Biotechnology Co. Ltd). The third experiment was set to detect sleep latency induced by barbital sodium (Lot #: 527B051, Merck).

TABLE 7
Study groups and dosages

| | | Dose | | |
|---|---|---|---|---|
| Group | | (mg/kg) | N | End point measurement |
| G1 | Vehicle | 0 | 15 | 1. Direct Sleep Test - no |
| G2 | Melatonin | 1 | 15 | pentobarbital |
| G3 | Zea mays (corn) leaf | 250 | 15 | 2. Sleep incidence - 32 |
| | extract standardized at | | | mg/kg Pentobarbital |
| | 6-MBOA | | | 3. Sleep time (Duration) - 46 |
| G4 | Chlorella-Low (L) | 1000 | 15 | mg/kg Pentobarbital |
| G5 | Chlorella-Mid (M) | 2000 | 15 | 4. Sleep onset latency - 240 |
| G6 | Chlorella-High (H) | 5000 | 15 | mg/kg Pentobarbital |
| G7 | Zea mays (corn) leaf | 250 + 1000 | 15 | |
| | extract standardized at | | | |
| | 6-MBOA + Chlorella-L | | | |
| G8 | Zea mays (corn) leaf | 250 + 2000 | 15 | |
| | extract standardized at | | | |
| | 6-MBOA + Chlorella-M | | | |
| G9 | Zea mays (corn) leaf | 250 + 5000 | 15 | |
| | extract standardized at | | | |
| | 6-MBOA + Chlorella-H | | | |

Zea mays (corn) leaf extract standardized at not less than 0.2% 6-MBOA (Lot #: FP210105-01) and Chlorella Powder (Lot #: 222A) were suspended with sterile water. Melatonin purchased from By-Health Co. Ltd. (Beijing, China) was used as a reference compound in each study. All mice were orally administrated with test materials in a volume of 20 mL/kg BW/day. Study lasted for 31 days.

SPSS software (Statistical Package for the Social Sciences, software for advanced statistical analysis) for windows was used for statistical analysis. The numbers of sleeping mice in the direct sleep test experiment and effect on sleep time under hypnotic subthreshold of pentobarbital sodium were analyzed by the χ2 test. Homogeneity between test groups were determined using variance analysis. If variance was not homogenous or when the data showed an abnormal distribution, an appropriate variable transformation was done to meet the requirements of homogeneity or normal distribution. When the variable transformation or the homogeneity was not yet reached after an appropriate variable transformation, the statistical method of rank test was used. The statistical method of t-test was used to analyze the changes in efficacy between melatonin and Zea mays (corn) leaf extract standardized at 6-MBOA.

### Example 9

### Test Material Showed No Drowsy Effect as Measured by a 60-Minute Direct Effect Test

In a direct effect test, mice will be considered sleeping and indexed as slept at the disappearance of reflex to turnover to the right side. Test was carried out an hour after administration of test materials. The principle of the test dictates, the recovery of turnover is defined as awakening, while the period between the disappearance and recovery of reflex to turnover to the right side is defined as the sleep time. The sleep time and numbers of mice who showed these characteristics were recorded and compared. It was considered to be positive if there was a difference in sleep time or numbers of mice sleeping between the tested substance groups and vehicle control.

TABLE 8
Direct sleeping effect of test materials.

| | | | | Sleep |
|---|---|---|---|---|
| | Dose | | Numbers of | Time |
| Groups | (mg/kg) | Numbers | sleeping | (min) |
| Vehicle | 0 | 15 | 0 | 0 |
| Melatonin | 1 | 15 | 0 | 0 |
| Zea mays (corn) leaf | 250 | 15 | 0 | 0 |
| extract standardized at 6- | | | | |
| MBOA | | | | |
| Chlorella-Low (L) | 1000 | 15 | 0 | 0 |
| Chlorella-Mid (M) | 2000 | 15 | 0 | 0 |
| Chlorella-High (H) | 5000 | 15 | 0 | 0 |
| Zea mays (corn) leaf | 250 + 1000 | 15 | 0 | 0 |
| extract standardized at 6- | | | | |
| MBOA + Chlorella-L | | | | |
| Zea mays (corn) leaf | 250 + 2000 | 15 | 0 | 0 |
| extract standardized at 6- | | | | |
| MBOA + Chlorella-M | | | | |
| Zea mays (corn) leaf | 250 + 5000 | 15 | 0 | 0 |
| extract standardized at 6- | | | | |
| MBOA + Chlorella-H | | | | |

As seen in the table 8, in the current study, all mice in each test material group immediately turn to their right side when they were placed in the supine position. This is an indication of no direct sleeping effect activity of test materials. Orally administering Zea mays (corn) leaf extract standardized at 6-MBOA, chlorella, and Zea mays (corn) leaf extract standardized at 6-MBOA+Chlorella to mice did not produce a drowsy effect in the absence of pentobarbital.

### Example 10

### Effect of Materials on Sleep Latency

Mice were intraperitoneally injected with 240 mg/kg barbital sodium (0.2 mL/20 g body weight), 15 min after the last test material administration. Sleep latency was recorded and the difference in sleep time between the tested substance groups and vehicle control were determined. A test material was considered positive (shortened sleep latency) when there was a difference in sleep onset time between the tested substance groups and vehicle control.

As shown in Table 9, sleep latency was significantly shortened for mice treated with 1 mg/kg melatonin. Similarly, mice received oral doses of 5,000 mg/kg chlorella showed statistically significant shorted sleep onset latency (p=0.024). The addition of Zea mays (corn) leaf extract standardized at 6-MBOA at 250 mg/kg to 5,000 mg/kg chlorella produced a further reduction in the latency of sleep (p=0.001) indicating the synergistic effect of these formulations. Additional analysis was carried out to evaluate the reduction in sleep onset latency observed for the melatonin (1 mg/kg) group compared against chlorella (5,000 mg/kg) alone and the combination of Zea mays (corn) leaf extract standardized at 6-MBOA and chlorella (250+5,000 mg/kg). It was only the composition of Zea mays (corn) leaf extract standardized at 6-MBOA and Chlorella with the statistical significance. This reduction in sleep latency further strengthens the added benefit of combining Zea mays (corn) leaf extract standardized at 6-MBOA and Chlorella.

TABLE 9
Synergistic effect of the composition on sleep onset latency

| | Dose | Num- | Latent Time | p- |
|---|---|---|---|---|
| Groups | (mg/kg) | bers | (min) | value |
| Vehicle | 0 | 15 | 49.7 ± 14.3 | — |
| Melatonin | 1 | 15 | 37.8 ± 12.8^{#} | 0.024 |
| Zea mays (corn) leaf | 250 | 15 | 44.0 ± 12.1 | 0.250 |
| extract standardized at 6- | | | | |
| MBOA | | | | |
| Chlorella-Low (L) | 1000 | 15 | 44.5 ± 16.0 | 0.635 |
| Chlorella-Mid (M) | 2000 | 15 | 39.2 ± 11.0 | 0.128 |
| Chlorella-High (H) | 5000 | 15 | 35.4 ± 16.0* | 0.024 |
| Zea mays (corn) leaf | 250 + 1000 | 15 | 43.1 ± 10.6 | 0.295 |
| extract standardized at 6- | | | | |
| MBOA + Chlorella-L | | | | |
| Zea mays (corn) leaf | 250 + 2000 | 15 | 41.9 ± 12.2 | 0.178 |
| extract standardized at 6- | | | | |
| MBOA + Chlorella-M | | | | |
| Zea mays (corn) leaf | 250 + 5000 | 15 | 33.0 ± 8.9*^{§} | 0.001 |
| extract standardized at 6- | | | | |
| MBOA + Chlorella-H | | | | |

There is a significant difference compared with vehicle control (0 mg/kgBW):
^{#}T test;
*analysis of variance.
^{§}Zea mays (corn) leaf extract standardized at 6-MBOA + Chlorella-H vs Melatonin.

### Example 11

### Combination of Zea mays (Corn) Leaf Extract Standardized at 6-MBOA to Chlorella Increased Incidence of Sleep

Mice were intraperitoneally injected with the hypnotic subthreshold dose (32 mg/kg) pentobarbital, 15 minutes post last dose administration of test materials to determine the incidence of sleep as a result of treatment effect. The numbers of mice that lost turnover to the right side over 1 min following injection of pentobarbital were recorded. The incidence of sleeping mice was analyzed to compare the difference between the tested substance groups and vehicle control. All the experiments were conducted at night.

TABLE 10
Synergistic effect to the combination of Zea mays (corn) leaf
extract standardized at 6-MBOA and Chlorella on sleep incidence

| | Dose | | Incidence | |
|---|---|---|---|---|
| Groups | (mg/kg) | Numbers | (%) | P value |
| Vehicle | 0 | 15 | 13 | — |
| Melatonin | 1 | 15 | 60^{#} | 0.021 |
| Zea mays (corn) leaf | 250 | 15 | 53* | 0.050 |
| extract standardized at 6- | | | | |
| MBOA | | | | |
| Chlorella-Low (L) | 1000 | 15 | 47 | 0.109 |
| Chlorella-Mid (M) | 2000 | 15 | 67* | 0.008 |
| Chlorella-High (H) | 5000 | 15 | 60* | 0.021 |
| Zea mays (corn) leaf | 250 + 1000 | 15 | 53* | 0.050 |
| extract standardized at 6- | | | | |
| MBOA + Chlorella-L | | | | |
| Zea mays (corn) leaf | 250 + 2000 | 15 | 60* | 0.021 |
| extract standardized at 6- | | | | |
| MBOA + Chlorella-M | | | | |
| Zea mays (corn) leaf | 250 + 5000 | 15 | 73* | 0.003 |
| extract standardized at 6- | | | | |
| MBOA + Chlorella-H | | | | |

There is a significant difference compared with vehicle control:
^{#}T test;
*analysis of variance

The addition of Zea mays (corn) leaf extract standardized at 6-MBOA to chlorella improved the incidence of mice falling to sleep in a dose-correlated manner. When mice were administered with chlorella alone at 1000 mg/kg, the incidence of mice falling to sleep was not statistically significant compared to vehicle, however, when Zea mays (corn) leaf extract standardized at 6-MBOA (250 mg/kg) was combined with chlorella (1000 mg/kg), the incidence of sleep was improved significantly compared to vehicle treated controls. Substantiating these finding, the formulation of Zea mays (corn) leaf extract standardized at 6-MBOA at 250 mg/kg and Chlorella at 5000 mg/kg, increased the sleep incidence time from 60% (sleep incident observed for chlorella alone was given at 5000 mg/kg) to 73%. These data signify the complementary effect of Zea mays (corn) leaf extract standardized at 6-MBOA and chlorella. In fact, the 73% sleep incidence for the combination of Zea mays (corn) leaf extract standardized at 6-MBOA (250 mg/kg) and chlorella (5000 mg/kg) was better than the 60% sleep incident observed for melatonin.

### Example 12

### Effect of Combination of Zea mays (Corn) Leaf Extract Standardized at 6-MBOA and Chlorella on Sleep Time

The amount of time spent sleeping was determined by injecting mice with 46 mg/kg pentobarbital sodium, intraperitoneally, 15 minutes after administration of the last dose of the test materials. The sleep time of each mouse was recorded and the difference of sleep time between tested materials and vehicle control were analyzed. As seen in Table 11, inverse relationship between sleep time and dosage was observed for mice treated with chlorella alone. The highest sleep time was observed for mice treated with lower dose of chlorella (1,000 mg/kg) followed by the mid dose (2,000 mg/kg). As the dose of chlorella increased, the duration of sleep was reduced. The addition of Zea mays (corn) leaf extract standardized at 6-MBOA to chlorella resulted in duration of sleeps equivalent to the melatonin treated mice for all the dosages. Zea mays (corn) leaf extract standardized at 6-MBOA by itself produced a duration of sleep similar to that of melatonin.

TABLE 11
Effect of combination of Zea mays (corn) leaf extract
standardized at 6-MBOA and Chlorella on sleep duration

| | Dose | Num- | Sleep Time | P |
|---|---|---|---|---|
| Groups | (mg/kg) | bers | (min) | value |
| Vehicle | 0 | 15 | 46.6 ± 11.6 | — |
| Melatonin | 1 | 15 | 59.7 ± 13.0^{#} | 0.007 |
| Zea mays (corn) leaf extract | 250 | 15 | 63.5 ± 10.8^{#} | 0.000 |
| standardized at 6-MBOA | | | | |
| Chlorella-Low (L) | 1000 | 15 | 70.0 ± 16.8* | 0.001 |
| Chlorella-Mid (M) | 2000 | 15 | 67.5 ± 18.6* | 0.002 |
| Chlorella-High (H) | 5000 | 15 | 61.6 ± 16.3* | 0.035 |
| Zea mays (corn) leaf extract | 250 + 1000 | 15 | 62.6 ± 12.2* | 0.009 |
| standardized at 6-MBOA + | | | | |
| Chlorella-L | | | | |
| Zea mays (corn) leaf extract | 250 + 2000 | 15 | 62.7 ± 14.4* | 0.009 |
| standardized at 6-MBOA + | | | | |
| Chlorella-M | | | | |
| Zea mays (corn) leaf extract | 250 + 5000 | 15 | 59.6 ± 18.1* | 0.041 |
| standardized at 6-MBOA + | | | | |
| Chlorella-H | | | | |

There is a significant difference compared with vehicle control:
^{#}T test;
*analysis of variance

### Example 13

### Preparation of a Sachet Powder Formula with the Combination of Chlorella and Zea mays (Corn) Leaf Extract Standardized at 6-MBOA

A combination of Chlorella and Zea mays (corn) leaf extract standardized at 6-MBOA powder were blended at a ratio of 9:1 with glucose powder, chicory root extract powder, skim milk powder, vegetable cream powder, sucralose. Granules are produced by wet granulation process with purified water as binder using an automatic system at inlet temp 55˜60° C., outlet temp 35˜40° C., and air injection pressure 26˜30 rpm. Granulated powder was further mixed with milk flavor powder and silicon dioxide to give 50% by weight of Chlorella: Zea mays (corn) leaf extract standardized at 6-MBOA=9:1 in the final granule powder mixture.

TABLE 12
Formula for Chlorella and Maizinol combination granule powder

| Materials | Weight ratio (%) |
|---|---|
| Chlorella:Maizinol (9:1) | 50.0 |
| Glucose powder | 20.9 |
| Chicory root extract powder | 10.0 |
| Skim milk powder | 10.0 |
| Vegetable cream powder | 5.0 |
| Milk flavor powder | 3.0 |
| Silicon dioxide | 1.0 |
| Sucralose | 0.1 |

### Example 14

### Preparation of a Tablet Formula with the Combination of Chlorella and Zea mays (Corn) Leaf Extract Standardized at 6-MBOA

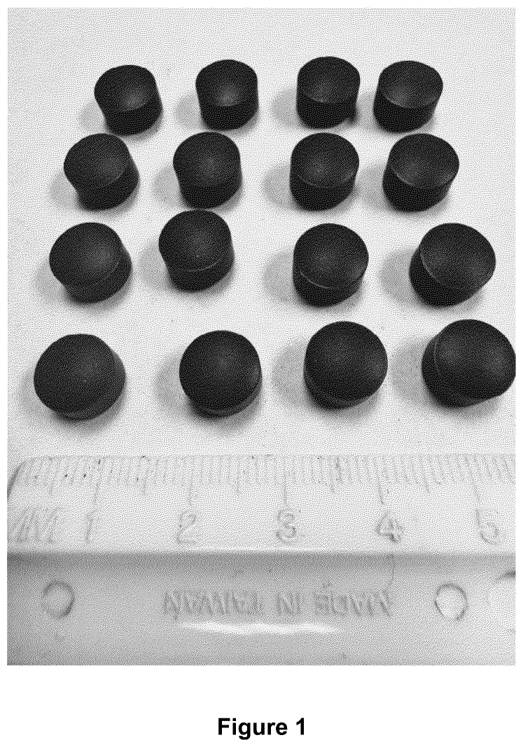

The first tablet formula example is described below, and the contemplated tablets are shown in FIG. 1. The above-mentioned tablet shown in FIG. 1 is designed for taking chlorella 2 g and Zea mays (corn) leaf extract standardized at 6-MBOA 250 mg per day. The above-mentioned tablet is designed for chlorella 333.333 mg and Zea mays (corn) leaf extract standardized at 6-MBOA 41.667 mg per tablet. The above-mentioned tablet formula is designed to take 6 tablets per day. The above-mentioned tablet is not designed to add any excipient such as diluting agent, but excipients can be added if needed.

The weight of the above-mentioned tablet is designed as 375 mg per tablet, and the blended amount of prototype was 2000 kg. Prototype production and inspection were commissioned to Japan tablet corporation (149-1 Megawa Makishima-cho Uji-City Kyoto Japan). Trial manufacturing Process is consisted of weighing, mixture and tablet making. The results of the prototype are described below.

The above-mentioned tablet thickness was 5.82 mm.

The above-mentioned tablet diameter was 9.10 mm.

The above-mentioned tablet hardness was 8.03 kgf.

The above-mentioned tablet weight was 375 mg.

The above-mentioned tablet disintegration was 16-18 min.

The above-mentioned tablet weight deviation was 0.41%.

The above-mentioned tablet moisture was 5.9%.

The above-mentioned tablet friability was 0.15%, which was 7.486 g before the test and was 7.475 g after the test.

The impact test results were acceptable.

The pilot scale tableting are conducted at a prototype production scale with mass production in mind, which have been confirmed that there are no problems with production scale tablet manufacturing.

The second tablet formula example is described below. The above-mentioned tablet is designed for taking chlorella 5 g and Zea mays (corn) leaf extract standardized at 6-MBOA 250 mg per day. The above-mentioned tablet is designed for chlorella 333.333 mg and Zea mays (corn) leaf extract standardized at 6-MBOA 16.667 mg per tablet. The above-mentioned tablet formula is designed to take 15 tablets per day. The above-mentioned tablet is not designed to add any excipient such as diluting agent, but excipients can be added if needed. The weight of the above-mentioned tablet is designed as 350 mg per tablet, and the blended amount of prototype was 2000 kg.

Prototype production and inspection were commissioned to Japan tablet corporation (149-1 Megawa Makishima-cho Uji-City Kyoto Japan). Trial manufacturing Process comprises weighing, mixture and tablet making. The results of the prototype are described below.

The above-mentioned tablet thickness was 5.57 mm.

The above-mentioned tablet diameter was 9.11 mm.

The above-mentioned tablet hardness was 5.9 kgf.

The above-mentioned tablet weight was 351 mg.

The above-mentioned tablet disintegration was 13-15 min.

The above-mentioned tablet weight deviation was 0.44%.

The above-mentioned tablet moisture was 6%.

The above-mentioned tablet friability was 0.01%, which was 7.027 g before the test and was 7.026 g after the test.

The impact test results were acceptable.

The pilot scale tableting are conducted at a prototype production scale with mass production in mind, which have been confirmed that there are no problems with production scale tablet manufacturing.

### Example 15

### Human Clinical Study of the Combination of Chlorella and Zea mays (Corn) Leaf Extract Standardized at 6-MBOA

The human clinical study can be initiated with a larger study population and additional biomarkers to substantiate the clinical study depicted in these examples. The clinical study protocol shall be provided with its entirety from the CRO.
CLINICAL DESIGN: A randomized, triple-blind, placebo controlled, parallel clinical trial to investigate the safety and efficacy of Investigational Product on sleep quality in a healthy population with difficulty falling asleep or staying asleep.
POPULATION: Healthy Adult Males & Females
SAMPLE SIZE: A sample size has been performed. 10-100 participants per group

### Clinical Trial Design Outcomes

Pittsburgh Sleep Quality Index (PSQI)
Profile of Mood States (POMS)
Perceived Stress Scale (PSS)
EEG or wearable device.
Actigraph readings: Sleep Duration, Sleep Onset, Wake Time, Circadian Consistency (0-100), Sleep Latency, Sleep Efficiency, Sleep Stages (awake, light, deep), Awakenings/Disruptions, Movement (w/intensity classification), Snoring (via cellular device microphone, w/intensity classification), Biometric trends: HR, HRV, SpO2, respiratory rate, arterial elasticity, Sleep Score (0-100), Recovery Score (0-100)
Blood markers: Serotonin, melatonin, GABA
Salivary markers: Cortisol
COVID-19 Impact on Quality of Life (QoL) Questionnaire

### Safety

Will be as assessed by: Clinical chemistry, hematology, vital sign as well as Adverse Events

### Inclusion Criteria

Males & females between 18 and 65 years of age
Females of childbearing potential must agree to use a medically approved method of birth
control and have a negative urine pregnancy test result
Unable to fall asleep or unable to stay asleep (2 or more waking episodes in a 7-day period)
Agrees to maintain current sleep schedule throughout the study.

Agrees to stay in the current time zone for the duration of the study
Agrees to refrain from over-the-counter (OTC) products to help with sleep.
Willingness to complete questionnaires, records and diaries associated with the study and to complete all clinic visits
Provided voluntary, written, informed consent to participate in the study

### Exclusion Criteria

Women who are pregnant, breast feeding, or planning to become pregnant during the trial
Alcohol or drug abuse in past year
Previous diagnosis of a sleep disorder
Current employment that calls for shift work
Currently experiencing nightmares and/or sleepwalking
Subject has a known allergy to the test material's active or inactive ingredients
Subjects with unstable medical conditions
Clinically significant abnormal laboratory results at screening
Participation in a clinical research trial within 30 days prior to randomization.
Individuals who are cognitively impaired and/or who are unable to give informed consent
Any other condition which in the Investigator's opinion may adversely affect the subject's
ability to complete the study or its measures or which may pose significant risk to the subject

### Treatments

Investigational Product—500 mg-5000 mg/day of the combination of Chlorella and Zea mays (corn) leaf extract standardized at 6-MBOA
Placebo

### Visit 1 (Screening):

Eligibility will be assessed and determined based on the inclusion and exclusion criteria. A urine pregnancy test will be performed (if applicable). Medical history and concomitant therapies will be reviewed; heart rate, and blood pressure will be measured. Peripheral blood will be collected to determine CBC, electrolytes (Na, K, CI), HbA1c, glucose, eGFR, creatinine, AST, ALT, ALP, and bilirubin.
Subjects will begin a 14-day run in period and complete Sleep diary every morning prior to baseline visit.

### Visit 2 (Baseline—Day 0):

Eligible participants will return to the clinic. Sleep diary will be collected and reviewed. Heart rate, and blood pressure will be measured; concomitant therapies will be reviewed. Subjects will be randomized into a treatment group. Pittsburgh Sleep Quality Index (PSQI), Perceived Stress Scale (PSS), the COVID-19 Impact on Quality of Life (QoL) Questionnaire and Profile of Moods (POMS) will be completed. Blood samples were collected to analyze Serotonin, melatonin, GABA. Saliva samples will be collected to measure cortisol levels. Participants will be provided with an Actigraphy device to wear on their wrist to monitor their sleep patterns at night and will be instructed to wear to bed each night. Participants will also be provided with an EEG device or a wearable device and will be trained for at-home use. Investigational product and subject treatment diary will be dispensed, and subjects will be instructed on use. The subject treatment diary will be used to record daily product use, changes in concomitant therapies and any adverse events and symptoms throughout the study.

### Visit 3 (Day 14):

Heart rate, and blood pressure will be measured. Investigational product and subject treatment diary will be returned, and compliance will be calculated. Concomitant therapies and adverse events will be reviewed. Sleep data will be collected from the Actigraphy device and EEG device. PSQI, PSS, the COVID-19 Impact on QoL Questionnaire and POMS will be completed. Blood samples were collected to analyse Serotonin, melatonin, GABA. Saliva samples will be collected to measure cortisol levels. The EEG device or the wearable device will be re-dispensed. Investigational product and subject treatment diary will also be re-dispensed.

### Visit 4 (Day 28—End of Study):

Heart rate and blood pressure will be measured. Investigational product and subject treatment diary will be returned, and compliance will be calculated. Concomitant therapies and adverse events will be reviewed. Sleep data will be collected from the Actigraphy device and EEG device. PSQI, PSS, the COVID-19 Impact on QoL Questionnaire and POMS will be completed. Blood samples will be collected to analyse Serotonin, melatonin, GABA. Saliva samples will be collected to measure cortisol levels. Blood samples will also be collected to determine CBC, electrolytes (Na, K, Cl), glucose, eGFR, creatinine, AST, ALT, ALP, and bilirubin.

### REFERENCES

1. Ahmad S B, Ali A, Bilal M, Rashid S M, Wani A B, Bhat R R, Rehman M U. Melatonin and Health: Insights of Melatonin Action, Biological Functions, and Associated Disorders. Cell Mol Neurobiol. 2023 August; 43(6):2437-2458.
2. Altman K and Greengard O (1966) Correlation of kynurenine excretion with liver tryptophan pyrrolase levels in disease and after hydrocortisone induction. J Clin Invest 45: 1527-1534.
3. Beverages 2021, 7(2), 33; https://doi.org/10.3390/beverages7020033
4. Comai S, Bertazzo A, Brughera M, Crotti S. Tryptophan in health and disease. Adv Clin Chem. 2020; 95:165-218. doi: 10.1016/bs.acc.2019.08.005.
5. Correia A S, Vale N. Tryptophan Metabolism in Depression: A Narrative Review with a Focus on Serotonin and Kynurenine Pathways. Int J Mol Sci. 2022 Jul. 31; 23(15):8493.
6. Davis I, Liu A. What is the tryptophan kynurenine pathway and why is it important to neurotherapeutics? Expert Rev Neurother. 2015; 15(7):719-21.
7. Guidance for Industry: Estimating the Maximum Safe Starting Dose in Initial Clinical Trials for Therapeutics in Adult Healthy Volunteer https://www.fda.gov/media/72309/download
8. Jovanovic F, Jovanovic V, Knezevic N N. Glucocorticoid Hormones as Modulators of the Kynurenine Pathway in Chronic Pain Conditions. Cells. 2023 Apr. 18; 12(8):1178.
9. Mar. Drugs 2022, 20(8), 493; https://doi.org/10.3390/md20080493
10. Nair A B, Jacob S. A simple practice guide for dose conversion between animals and human. J Basic Clin Pharm. 2016 March; 7(2):27-31.
11. Nutrients 2023, 15(9), 2168; https://doi.org/10.3390/nu15092168.
12. Pourhamzeh M, Moravej F G, Arabi M, Shahriari E, Mehrabi S, Ward R, Ahadi R, Joghataei M T. The Roles of Serotonin in Neuropsychiatric Disorders. Cell Mol Neurobiol. 2022 August; 42(6):1671-1692.
13. Sutanto C N, Loh W W, Kim J E. The impact of tryptophan supplementation on sleep quality: a systematic review, meta-analysis, and meta-regression. Nutr Rev. 2022 Jan. 10; 80(2):306-316.

1. A composition, comprising a plant extract standardized with an amount of benzoxazinoids, wherein the composition additionally comprises Chlorella.

2. The composition of claim 1, wherein the composition is provided in an effective amount to shorten sleep onset latency, improve sleep quality and efficiency, and prevent and treat sleep disorders.

3. The composition according to claim 1, wherein the plant extract standardized with an amount of benzoxazinoids is extracted, enriched, and standardized from a plant species selected from the group comprising Zea mays, Chlorella sorokiniana, Chlorella vulgaris, Oryza species, Oryza sativa, Oryz glaberrima, Oryz australiensis, Oryz brachyantha, Secale cereale, Acanthus arboreus; Acanthus ebracteatus, Acanthus illicifolius, Acanthus mollis, Avena sativa, Avena abyssinica, Avena byzantine, Avena nuda, Avena strigosa, Hordeum vulgare, Coix lachryma-jobi, Triticum aestivum, Triticum compactum, Triticum sphaerococcum, Triticum turanicum, Sorghum bicolor, Agropyron repens, Blepharis edulis, Balsamocitrus paniculate; Peristrophe roxburghiana; Strobilanthes cusia; Lamium galeobdolon, Lobelia chinensis, Leymus chinensis, Aphelandra spp, Scoparia dulcis, Capparis sikkimensis ssp, or a combination thereof.

4. The composition according to claim 1, wherein the plant extract standardized with an amount of benzoxazinoids is extracted, enriched and standardized from a plant part selected from the group comprising shoots, germinates from plant seeds, sprouts of geminated grains, immature leaves, mature leaves, whole plants, roots, seeds, flowers, stems, stem barks, root barks, silk, grain, hair roots of germinated grain, stem cells, cell culture tissues, or any combination thereof.

5. The composition according to claim 1, wherein the benzoxazinoids are one or more compounds of 6-Methoxy-2-benzoxazolol (6-MBOA); 2-Benzoxazolol (2-BOA); 4-Methylbenzoxazole; 2,4-Dimethylbenzoxazole; 2,6-Dimethylbenzoxazole; 2,6-Benzoxazolediol; 2,4-Benzoxazolediol; 4-Acetyl-2(3H)-benzoxazolone; 6-Methoxy-N-methyl-2(3H)-benzoxazolone; 3-Hydroxy-6-methoxy-2-benzoxazolin-2(3H)-one; 2-Hydroxy-6,7-dimethoxybenzoxazole; 5,6-Dimethoxy-2-benzoxazolinone; 3,6-Dimethoxybenzoxazolin-2(3H)-one; 5-Chloro-6-methoxy-2-benzoxazolinone; Trehalamine or any combination thereof and benzoxazinoid glycosides.

6. The composition according to claim 1, wherein the plant extract standardized with an amount of benzoxazinoids is extracted with any suitable solvent, including supercritical fluid of CO2, water, acidic water, basic water, acetone, methanol, ethanol, propenol, butanol, alcohol mixed with water, mixed organic solvents or a combination thereof.

7. The composition according to claim 1, wherein the benzoxazinoids are synthesized, metabolized, biodegraded, bioconverted, biotransformed, biosynthesized from small carbon units, by transgenic microbial, by P450 enzymes, by glycotransferase enzyme or a combination of enzymes, by microbacteria.

8. The composition according to claim 1, wherein the plant extract standardized with an amount of benzoxazinoids is enriched individually or in combination by solvent precipitation, neutralization, solvent partition, ultrafiltration, enzyme digestion, column chromatograph with silica gel, XAD, HP20, LH20, C-18, alumina oxide, polyamide, ion exchange and CG161 resins.

9. The composition according to claim 1, wherein the plant extract standardized with an amount of benzoxazinoids is extracted, enriched and standardized from shoots and immature leaves of Zea mays containing not less than 0.2% 6-MBOA.

10. The composition according to claim 1, wherein the Chlorella is CK-22 cultured and sequentially expanded in slant culture, flask culture, jar culture, seed culture, tank culture, and finally, outdoor pool culture.

11. The composition according to claim 1, wherein the Chlorella is CK-22 outdoor poor cultured to process by washing, filtering, sterilizing, and spray drying.

12. The composition according to claim 1, wherein the Chlorella particle distribution of the Chlorella powder is in a range of 1 μm to 500 μm.

13. The composition according to claim 1, wherein the Chlorella contains Vitamin B2 not less than 4 mg/100 gram, proteins not less than 55%, tryptophane not less than 1 gram/100 grams, and iron not less than 40 mg/100 gram.

14. The composition according to claim 1, wherein the plant extract standardized with an amount of benzoxazinoids is in combination with chlorella in a blending ratio range of 0.05%:99.95% to 99.95%:0.05% by weight.

15. The composition according to claim 1, wherein the composition further comprises a pharmaceutically or nutraceutically or functional food acceptable active, adjuvant, carrier, diluent, or excipient, wherein the pharmaceutical or nutraceutical formulation comprises from about 0.1 weight percent (wt %) to about 99.9 wt % of the composition in the enriched one or more benzoxazinoids.

16. The composition of claim 15, wherein the active, adjuvant, excipient or carrier is selected from one or more of Cannabis sativa oil or CBD/THC, turmeric extract or curcumin, terminalia extract, Aloe vera leaf gel powder, Valerian roots, Valeriana officinalis, Ginkgo biloba, Kava kava, Lavender, Passionflower (Passiflora incarnata or maypop), Chamomile flower, Hops, Humulus lupulus, Hibiscus sabdariffa, St. John's Worth, Calif Griffonia simplicifolia, Fermented milks, fish oil, Rhodiola rosea, Lotus seed, Lotus seed germ, Oryza sativa, Zea mays, Chlorella sorokiniana, Chlorella vulgaris, other Chlorella spp, Ziziphus jujuba, Schisandra chinensis, Magnolia officinalis, Astragalus membranaceus, Ganoderma lucidum, Echinacea purpurea, Echinacea angustifolia, Poria cocos Wolf, Wolfiporia extensa, Withania somnifera, Bupleurum falcatum, Glycyrrhiza spp, Panax quinquefolium, Panax ginseng C. A. Meyer, Korea red ginseng, Eurycoma longifolia (Malaysian ginseng) Lentinula edodes (shiitake), Inonotus obliquus (Chaga mushroom), Melatonin, Magnesium, gamma aminobutyric acid (GABA), vitamin B1, B2, B3, B6, B12, Pyridoxine, Methylcobalamin, Niacinamide, Folic acid, Ascorbic acid, vitamin C, Vitamin D & E, Zinc, omega-3 fatty acid, Glycine, Glutamine, arginine, Tryptophan, L-theanine, 5-Hydroxytryptophan (%-(5-HTP), SAMe, Magnolol, Honokiol, Taurine, Boron, Branched-Chain amino acids (BCAA), phospholipids, phosphatidylserine, phosphatidic acid, theaflavin, rosmarinic acid, catechin, epicatechin, conjugated catechins such as EGCG, ECG, epigallocatechin etc. baicalein, baicalin, Oroxylin, Wogonin, Kaempferol, genistein, quercetin, Butein, Betaine, Luteolin, chrysin, Apigenin, curcumin, resveratrol, glomeratose A, 6-shogaol, gingerol, berberine, piperine.

17. The composition according to claim 1, wherein the composition is formulated as a tablet, hard capsule, soft gel capsule, powder, or granule, compressed tablet, pill, gummy, chewing gum, sachet, wafer, bar, or liquid form, tincture, aerial spread, semi solid, semi liquid, solution, emulsion, cream, lotion, ointment, gel base or like form.

18. The method according to claim 1, wherein the route of the administration is selected from the group comprising oral, topical, suppository, intravenous, intradermic, intragastric, intramuscular, intraperitoneal, and intravenous.

19. The composition according to claim 1, wherein the plant extract standardized with an amount of benzoxazinoids in combination with Chlorella prevents and treats sleep disorders.

20. The composition of claim 19, wherein the sleep disorders include insomnia, hypersomnia, circadian rhythm disorders, shift work sleep disorder, non-24-hour sleep-wake disorder, periodic limb movement disorder, restless legs syndrome (RLS), sleep apnea, narcolepsy, Parasomnias, night terrors, sleepwalking, nightmares, Sleep Eating Disorder, sleep Hallucinations, Sleep Paralysis, Sleep Talking, REM Sleep Behavior Disorder.

21. The composition according to claim 1, wherein the plant extract standardized with an amount of benzoxazinoids in combination with Chlorella shortens sleep onset latency, improves sleep quality and efficiency, increases deep sleep time, increases total sleep time, improves REM and NREM sleep.

22. The composition according to claim 1, wherein the plant extract standardized with an amount of benzoxazinoids in combination with Chlorella maintains homeostasis of cortisol that leads to improved symptoms of chronically high cortisol.

23. The composition of claim 22, wherein symptoms of chronically high cortisol include anxiety, depression, fatigue, gastrointestinal upset like constipation, bloating, or diarrhea, headache, heart disease, high blood pressure, irritability, problems with memory and concentration, reproductive issues like low libido, erectile dysfunction, or irregular menstruation and ovulation, sleep difficulties, slow recovery from exercise, eating disorders and weight gain, hair loss, chronic inflammation, susceptibility to infection and skin blemishes.

24. The composition according to claim 1, wherein the plant extract standardized with an amount of benzoxazinoids in combination with Chlorella is administered at an effective amount of from 0.01 mg/kg to 5,000 mg/kg body weight of the mammal.

25. The composition of claim 24, wherein the mammal is human, companion animals, farm and wild animals including cats, dogs, swine, cattle, sheep, goats, horses, and rabbits.
